(19) United States
(12) Patent Application Publication
(10) Pub. No.: US 2013/0239660 A1
(43) Pub. Date: Sep. 19, 2013

(54) Environment Sampling System

(71) Applicants: James P. Gunderson, Denver, CO (US); Mark C. Vappi, Denver, CO (US)

(72) Inventors: James P. Gunderson, Denver, CO (US); Mark C. Vappi, Denver, CO (US)

(21) Appl. No.: 13/847,470

(22) Filed: Mar. 19, 2013

Related U.S. Application Data
(60) Provisional application No. 61/612,765, filed on Mar. 19, 2012.

(51) Int. Cl. G01N 33/00

(52) U.S. Cl. CPC G01N 33/0004
USPC 73/31.01

(57) ABSTRACT

An environmental sampling system for use with a mobile support device, the environmental sampling system includes a body with a surrounding sidewall that is about a longitudinal axis to define a body interior, further included is a sensor disposed therethrough the surrounding sidewall, wherein the sensor is in fluid communication with the interior. Also, an inlet portion is adjacent to the surrounding sidewall, wherein the inlet portion is in fluid communication with the interior, and an outlet portion that is adjacent to an opposing end of the surrounding sidewall, wherein the outlet portion is in fluid communication with the interior, and a means for initiating gas movement therethrough the inlet portion, the body, and the outlet portion is provided to enhance the sensor gas contaminant detection performance.

### RELATED PATENT APPLICATION

This is a continuation in part (CIP) patent application that claims benefit of U.S. provisional patent application Ser. No. 61/612,765 filed on Mar. 19, 2012 by James P. Gunderson and Mark C. Vappi of Denver, Colo., U.S.

### TECHNICAL FIELD

The present invention relates generally to a system for testing atmospheric gas. More specifically, the present invention relates to the field of automated environmental gas sensing utilizing a mobile apparatus such as a robot for the functional purpose of monitoring atmospheric gas conditions throughout a defined volumetric space.

### BACKGROUND OF INVENTION

As robots are becoming more and more common for accomplishing everyday tasks, robots are particularly well suited for mundane or repetitive operations or functions that need to be performed in hazardous areas. As it is known for robots to incorporate video and audio to virtually have the eyes and ears of a human to allow for remote visual and audio perception in places humans cannot easily go-such as deep underwater or in dangerous areas due to explosives present or from a toxic environmental situation, it follows that the human sense of smell would next be incorporated into the robots abilities. Thus adding a “nose” to a robot would further enhance the robots capabilities for inspecting and report/tracking the environmental substances present without the need for human presence in a dangerous area-plus with the benefit of mobility that a robot can provide for environmental sensing in multiple locations. This as opposed to putting environmental sensing equipment in a fixed location or having to add multiple sets of environmental sensing equipment to accommodate multiple location environmental sensing capabilities. Wherein with a single environmental sensor the mobile robot can sense environmental conditions in selected multiple locations due to the robots mobility.

What is needed is a portable and mobile environmental sensing system that is designed to work with a travelling robot for the purpose of detecting for example; carbon monoxide, carbon dioxide, temperatures, volatile or explosive gases, smoke, humidity levels, and the like—thus being able to monitor specific selected areas of for instance inside of a warehouse, wherein changes in environmental conditions would be of interest.

### SUMMARY OF INVENTION

Broadly, the present invention is an environmental sampling system that is designed to integrate with a mobile robot to be able to have the functional capability to monitor the environment for potential hazards in selected multiple locations. Typical uses would include patrolling garage areas for smoke, carbon monoxide buildup, or for explosive gases. Other uses would include monitoring indoor facilities for potential problems such as humidity buildup, excess temperature, and carbon dioxide buildup in wineries. Added uses include radiation detection and other airborne hazards.

These and other objects of the present invention will become more readily appreciated and understood from a consideration of the following detailed description of the exemplary embodiments of the present invention when taken together with the accompanying drawings, in which;

### BRIEF DESCRIPTION OF DRAWINGS

FIG. 1 shows a side elevation view of the environmental sampling system, including the inlet portion, the gas movement, the sensing chamber body, the longitudinal axis for the body, the sensors, the outlet control valve, and the outlet portion;

FIG. 2 shows a perspective view of the environmental sampling system, including the inlet portion, the gas movement, the sensing chamber body, the longitudinal axis for the body, the sensors, the outlet control valve, and the outlet portion;

FIG. 3 shows a perspective view of the environmental sampling system, including a cross section of the inlet portion with a filter shown disposed within the inlet, the gas movement, the sensing chamber body, the longitudinal axis for the body, the sensors, and a cross section of the outlet portion showing the outlet control valve, and the outlet portion;

FIG. 4 shows cross section cut 4-4 from FIG. 3 showing a cross section view of the environmental sampling system, including a cross section of the inlet portion with a filter shown disposed within the inlet, the gas movement, the means for initiating gas movement disposed within the inlet portion, the sensing chamber body cross section, the longitudinal axis for the body, the sensors disposed within the body surrounding sidewall, a sensor mounting flange, a sensor electrical communication, and a cross section of the outlet portion showing the outlet control valve, and the outlet portion;

FIG. 5 shows cross section cut 5-5 from FIG. 4 showing a cross section view of the environmental sampling system, including a cross section of the inlet portion with a filter shown disposed within the inlet, the gas movement, the means for initiating gas movement disposed within the inlet portion, the sensing chamber body cross section, the longitudinal axis for the body, the sensors disposed within the body surrounding sidewall, a sensor mounting flange, a sensor electrical communication, and a cross section of the outlet portion showing the outlet control valve, and the outlet portion, also a controller, a controller power input, a controller data signal output, and the sensor electrical communication; and

FIG. 6 shows a performance curve for the means for initiating gas movement having an X axis scale of increasing interior flow rate from left to right and a Y axis scale of increasing interior pressure from bottom to top, with movement along the curve shown.

### REFERENCE NUMBERS IN DRAWINGS

50 Environment Sampling System
51 Atmospheric area
52 Atmospheric gas
53 Gas 52 turbulence (non-laminar flow) at sensor 85
55 Inlet portion, atmospheric gas 52
60 Filter, inlet
65 Movement of gas 52
70 Body, sensing chamber
75 Surrounding sidewall of the body 70
76 Interior of the body 70
80 Longitudinal axis for the body 70
85 Sensors for various environmentally borne substances in the gas 52
90 Means for initiating gas 52 movement 65 through the inlet portion 55, the body 70, and the outlet portion 100
95 Fan that is an in-line axial direct drive type for the means 90 for initiating gas 52 movement 65
96 Performance curve of the fan 95 for the gas 52
97 Moving along the performance curve 96
100 Outlet portion, atmospheric gas 52
104 Means to regulate gas 52 movement
105 Outlet control valve for the means 104 to regulate gas 52 movement
110 Mounting saddle support flange for sensor 85
115 Electrical communication from the sensor 85
120 Controller
125 Power input for controller 120
130 Data signal output for controller 120
135 Sensor 85 input for the controller 120
140 Axial axis of rotation of the fan 95
145 Rotational direction of the fan 95

### DETAILED DESCRIPTION

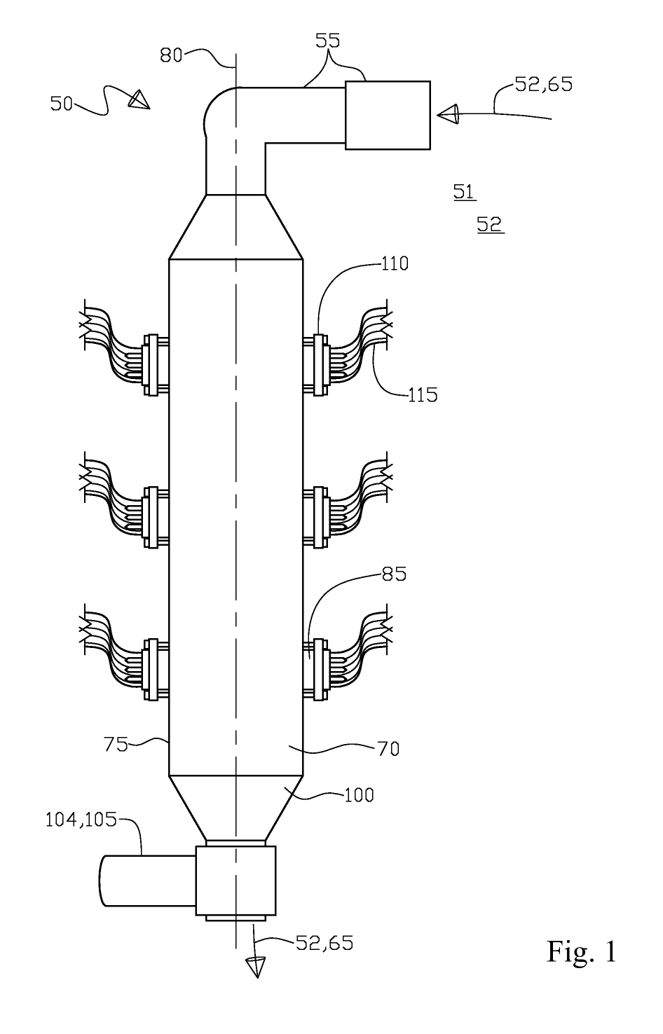

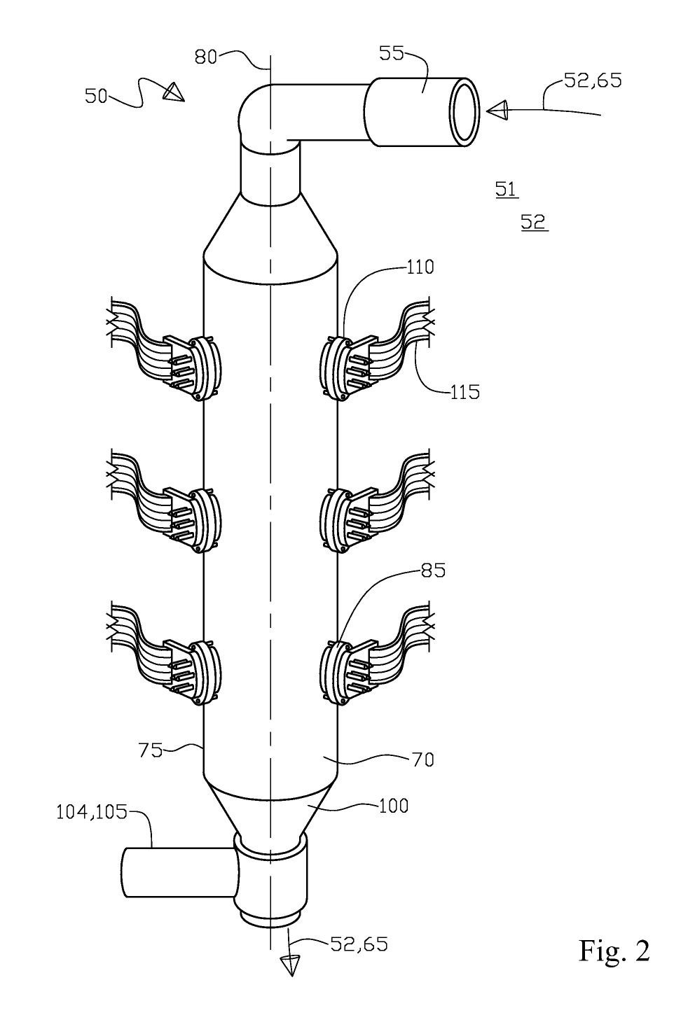

With initial reference to FIG. 1 shown is a side elevation view of the environmental sampling system 50, including the inlet portion 55, the gas movement 65, the sensing chamber body 70, the longitudinal axis 80 for the body 70, the sensors 85, the outlet control valve 105 and the outlet portion 100. Continuing, FIG. 2 shows a perspective view of the environmental sampling system 50, including the inlet portion 55, the gas movement 65, the sensing chamber body 70, the longitudinal axis 80 for the body 70, the sensors 85, the outlet control valve 105 and the outlet portion 100.

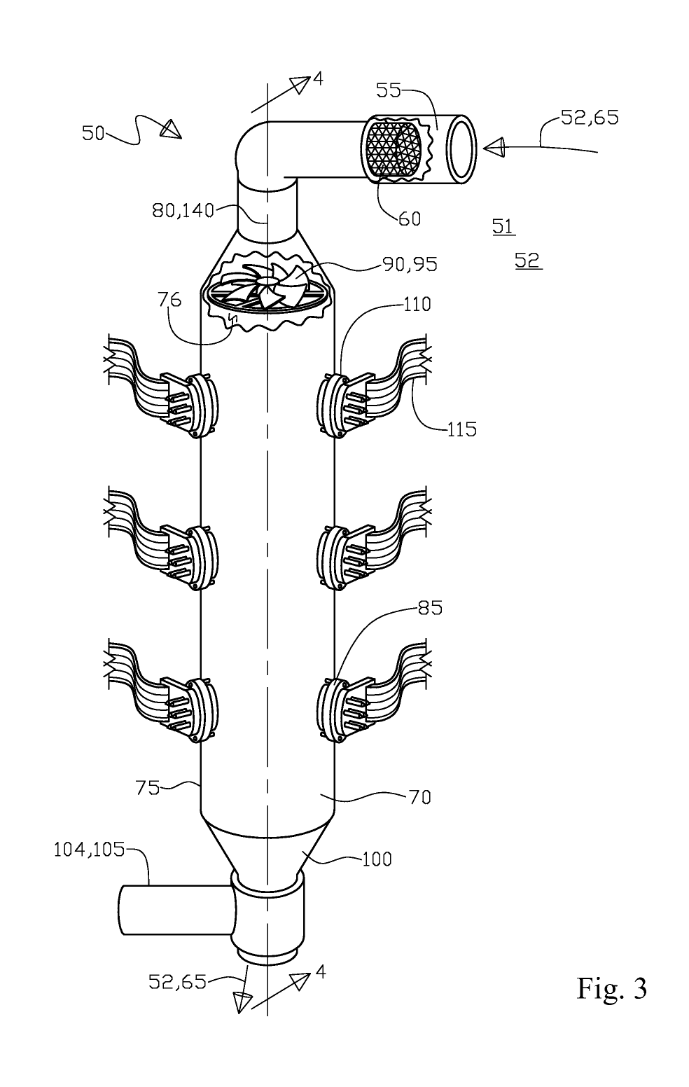

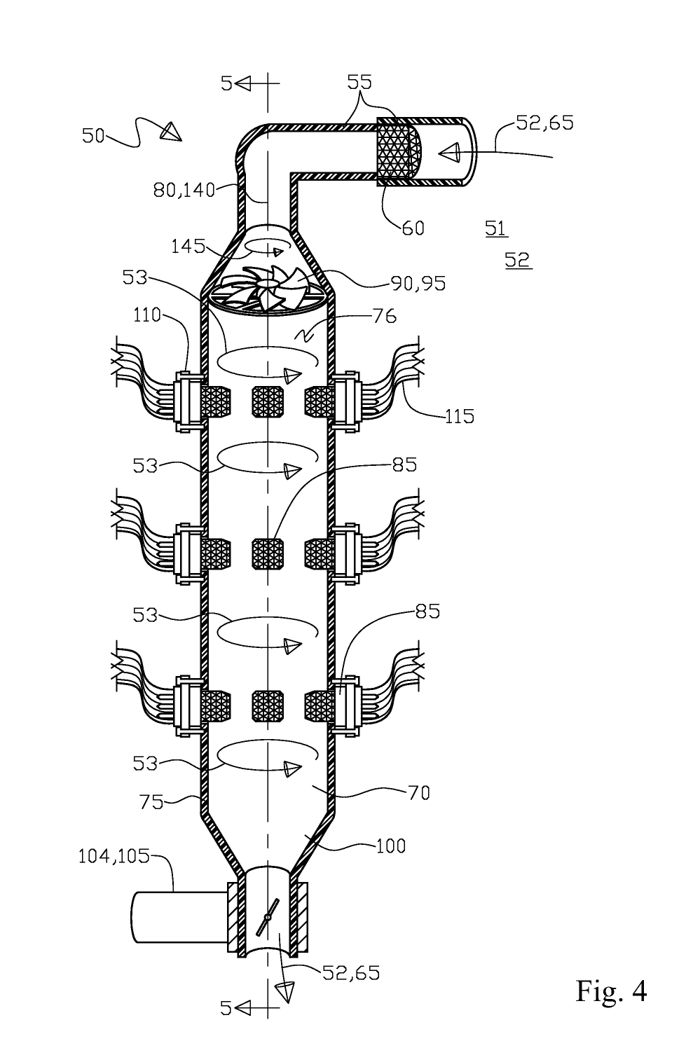

Further, FIG. 3 shows a perspective view of the environmental sampling system 50, including a cross section of the inlet portion 55 with a filter 60 shown disposed within the inlet portion 55, the gas movement 65, the sensing chamber body 70, the longitudinal axis 80 for the body 70, the sensors 85, and a cross section of the outlet portion 100 showing the outlet control valve 105, and the outlet portion 100. Next, FIG. 4 shows cross section cut 4-4 from FIG. 3 showing a cross section view of the environmental sampling system 50, including a cross section of the inlet portion 55 with a filter 60 shown disposed within the inlet portion 55. FIG. 4 also shows the gas movement 65, the means 90 for initiating gas movement 65 disposed within the inlet portion 55, the sensing chamber body 70 cross section, the longitudinal axis 80 for the body 70, the sensors 85 disposed within the body 70 surrounding sidewall 75. In addition, FIG. 4 shows the sensor mounting flange 110, the sensor electrical communication 115, and a cross section of the outlet portion 100 showing the outlet control valve 105, and the outlet portion 100.

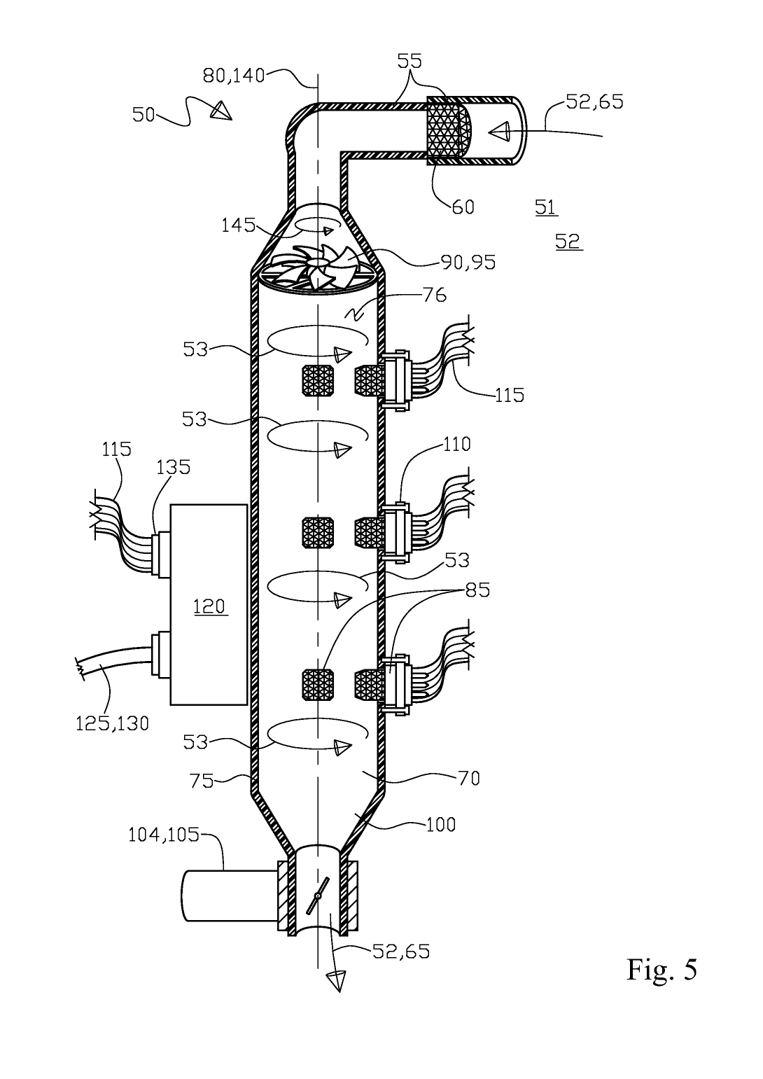

Continuing, FIG. 5 shows cross section cut 5-5 from FIG. 4 showing the cross section view of the environmental sampling system 50, including a cross section of the inlet portion 55 with the filter 60 shown disposed within the inlet 55, the gas 52 movement 65, the means 90 for initiating gas 52 movement 65 disposed within the inlet portion 55. Further, FIG. 5 shows the sensing chamber body 70 cross section, the longitudinal axis 80 for the body 70, the sensors 85 disposed within the body 70 surrounding sidewall 75, a sensor 85 mounting flange 110, a sensor 85 electrical communication 115. Additionally, FIG. 5 shows the cross section of the outlet portion 100 showing the outlet control valve 105, and the outlet portion 100, also a controller 120, a controller power input 125, a controller data signal output 130, and the sensor 85 electrical communication 115.

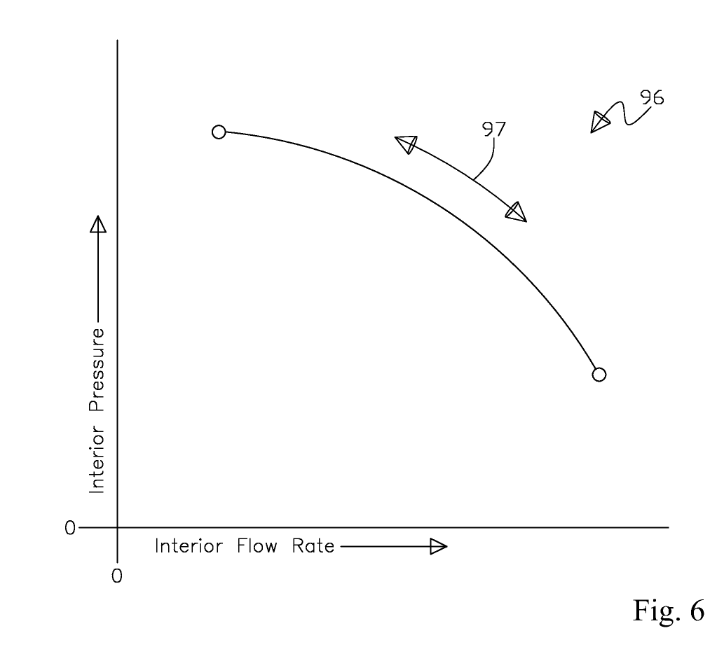

Further, FIG. 6 shows a performance curve 96 for the means 90 for initiating gas movement having a X axis scale of increasing interior 76 flow rate from left to right and a Y axis scale of increasing interior 76 pressure from bottom to top, with movement 97 along the curve 96 shown.

Broadly speaking, as shown in FIGS. 1 to 5, the environmental sampling system 50 is for use with a mobile support device that would preferably be a travelling robot; the environmental sampling system 50 includes the body 70 with the surrounding sidewall 75 being about the longitudinal axis 80 to define the body 70 interior 76. Further included in the environmental sampling system 50 is the sensor 85 that is disposed therethrough the surrounding sidewall 75, wherein the sensor 85 is in fluid communication with the interior 76, as best shown in FIG. 4. Also included is the inlet portion 55 that is adjacent to the surrounding sidewall 75, wherein the inlet portion 55 is in fluid communication with the interior 76, again as best shown in FIG. 4. Further included in the environmental sampling system 50 is the outlet portion 100 that is adjacent to the opposing end of the surrounding sidewall 75, wherein the outlet portion 100 is in fluid communication with the interior 76, again as best shown in FIG. 4. In addition, included is the means 90 for initiating gas movement 65 that is preferably disposed in the inlet portion 55, also the body 70, and the outlet control valve 105 is shown that is disposed in the outlet portion 100, as shown in FIGS. 3 and 4.

Also, the sensors 85 are mounted to the sidewall 75 via a flange 110 with the electrical communication 115 for the sensor 85 oppositely located in the atmospheric area 51, as shown in FIG. 4. Further in FIG. 5, the controller 120 has a sensor 85 input 135 via the electrical communication 115, further the controller 120 has a power input 125, and a data signal output 130.

The environment sampling system 50 unit, also known as the “nose”, is designed to integrate into the mobile support device which is preferably a VIGILUS security robot. The environment sampling system 50 includes the inlet portion 55, a replaceable inlet filter 60, a body 70 that has a surrounding sidewall 75 that is about a longitudinal axis 80, to define a body 70 and surrounding sidewall 75 interior 76, and an outlet portion 100. Disposed therethrough the surrounding sidewall 75 is a sensor 85 for measuring gas 52 borne contaminants that are present in the gas 52 that is disposed within the interior 76. Note that there could be a plurality of sensors 85 for detecting multiple different types of gas 52 contaminants.

Gas 52 is drawn into the interior 76 via the inlet portion 55 and through the filter 60 by the means 90 for initiating gas movement 65 through the interior 76 and exhausting through the outlet portion 100 through the outlet control valve 105, wherein the control valve 105 is modulated to control the gas 52 that is disposed within the interior 76 for varying the gas 52 flow rate therethrough the interior 76 and for varying the gas 52 pressure within the interior 76, essentially moving back and forth 97 along the outlet pressure-flow curve of the fan 95, see FIG. 6, thus resulting in the interior 76 gas 52 pressure always being greater than the gas 52 atmospheric 51 pressure—thus controlling the gas 52 dwell time, flow rate, and pressure that the sensor 85 is exposed to in the interior 76, as desired for sensor 85 optimal performance in detecting gas 52 contaminants. Thus the means 90 is preferably a software controlled fan 95 that is to ensure gas 52 sample densities and flow rates in the interior 76 that each individual sensor 85 are exposed to are controlled.

Again, broadly speaking, as shown in FIGS. 1 to 6, the environment sampling system 50 for use with a mobile support device, the environment sampling system 50 includes the body 70 that includes the surrounding sidewall 75 that is about the longitudinal axis 80 to define the body 70 interior 76 as opposed or distinguished to an atmospheric area 51 that is external to the interior 76. Further included in the environment sampling system 50 is the sensor 85 disposed therethrough the surrounding sidewall 75, wherein the sensor 85 is in fluid communication with interior 76, also included is the inlet portion 55 that is adjacent to the surrounding sidewall 75, wherein the inlet portion 55 is in fluid communication with the interior 76. The environment sampling system 50 also has the outlet portion 100 that is adjacent to the opposing end of the surrounding sidewall 75, wherein the outlet portion 100 is in fluid communication with the interior 76, also there is the means 90 for initiating gas 52 movement therethrough the inlet portion 55, the interior 76, and the outlet portion 100, as best shown in FIG. 4.

Looking in particular at FIGS. 4, 5, and 6, for the environment sampling system 50, wherein the means 90 for initiating gas 52 movement 65 is preferably constructed of an in-line axial direct drive type fan 95, wherein the fan has an axial axis 140 of rotation 145, see FIGS. 4, 5, and 6. In addition, the fan 95 is preferably disposed within the interior 76 being positioned such that the fan axial axis 140 of rotation 145 is coincident to the longitudinal axis 80, see FIGS. 4 and 5, with the fan 95 being operational to initiate gas 52 turbulence 53 at the sensor 85 to enhance sensor 85 performance in detecting atmospheric or environmental borne substances.

Also, for the environment sampling system 50, preferably the sensor 85 further comprises a saddle support flange 110 interface to support the sensor 85 to mount therethrough the sidewall 75, see FIGS. 4 and 5.

As an enhancement for the environment sampling system 50 it can preferably further comprise a means 104 to regulate the gas 52 movement 65 via a gas 52 pressure change and a gas 52 flow rate change. Preferably, the means 104 to regulate the gas 52 movement 65 is a control valve 105 having an adjustable range from in-between a fully closed operational state to a fully open operational state, see FIGS. 4 and 5, wherein the control valve 105 is shown positioned between the fully closed operational state to the fully open operational state, also see FIGS. 1, 2, and 3, for the exterior of the control valve 105.

Further, on the control valve 105, it is preferably disposed within the outlet portion 100 being positioned downstream, in relation to the gas 52 movement 65 from the means 90 for initiating gas 52 movement 65, this is to operationally regulate the gas 52 movement 65 via the gas 52 pressure changes and the gas 52 flow rate changes within the interior 76 via moving along 97 a performance outlet curve 96 of the means 90 for initiating gas 52 movement 65, as required for the sensor 85 to have enhanced gas 52 contaminant detection performance by regulating the control valve 105 between the fully closed state and the fully open state, see in particular FIGS. 4, 5, and 6.

In addition, on the environment sampling system 50, it can preferably further comprise a controller 120 that is in electrical communication 115 with the sensor 85, see FIG. 5, wherein operationally the controller 120 converts an analog signal input 115 from the sensor 85 into a digital output 130 signal, note that the controller 120 also has a power input 125. Further, on the controller 120, it is preferably positionally affixed to the sidewall 75 in the atmospheric area 51 to minimize distance of the sensor electrical communication 115, wherein a plurality of sensors 85 could be disposed therethrough the sidewall 75, see FIG. 5.

The individual sensors 85 are analyzed by proprietary software running on a dedicated 32-bit, 160 MIPS microcontroller, and the results are available via USB or 12C communications, depending upon the requirements of the mobile support device.

The environmental sampling system 50 is compatible with all models of the VIGILUS security robot and is capable of either consistent or on demand gas sampling by the sensors 85. Wherein the sensors 85 typically test for gases that include carbon monoxide, explosive or volatile gases, smoke, carbon monoxide, humidity levels, temperature levels, radiation levels, and the like. Thus, the environmental sampling system 50 is designed to integrate into the VIGILUS security robot to have the ability to monitor the environment for potential hazards. Typical applications would include patrolling garage areas for smoke, carbon monoxide buildup, or explosive gases. Other uses could include monitoring indoor facilities for potential problems such as humidity build up, excess temperature, and the carbon dioxide buildup in wineries.

Standard equipment for the environmental sampling system 50 would include an integrated 32-bit 160 MIPS 8 core microcontroller for sample analysis, a 0.5 ft.³ per minute flow control fan 95, replaceable inlet filters 60, USB and 12C communications, and a self calibration mode.

Specifications for the environmental sampling system 50 are approximately;
Power—12 V DC
Power consumption—250 milliamps plus approximately 20 milliamps per sensor 85
Preferred maximum sensors 85 support is 20 in quantity
Processor—an onboard 32-bit 160 MIPS 8 core
Weight—7 pounds
Height—23 inches
Width—8 inches inlet portion 55 to sensor 85 clearance

### CONCLUSION

Accordingly, the present invention of an Environmental Sampling System has been described with some degree of particularity directed to the embodiments of the present invention. It should be appreciated, though; that the present invention is defined by the following claims construed in light of the prior art so modifications or changes may be made to the exemplary embodiments of the present invention without departing from the inventive concepts contained therein.

1. An environment sampling system for use with a mobile support device, said environment sampling system comprising:
(a) a body that includes a surrounding sidewall about a longitudinal axis to define a body interior as opposed to an atmospheric area;
(b) a sensor disposed therethrough said surrounding sidewall, wherein said sensor is in fluid communication with said interior;
(c) an inlet portion adjacent to said surrounding sidewall, wherein said inlet portion is in fluid communication with said interior;
(d) an outlet portion adjacent to an opposing end of said surrounding sidewall, wherein said outlet portion is in fluid communication with said interior; and
(e) a means for initiating gas movement therethrough said inlet portion, said interior, and said outlet portion.

2. An environment sampling system according to claim 1 wherein said means for initiating gas movement is constructed of an in-line axial direct drive type fan, wherein said fan has an axial axis of rotation.

3. An environment sampling system according to claim 2 wherein said fan is disposed within said interior being positioned such that said fan axial axis of rotation is coincident to said longitudinal axis, with said fan being operational to initiate gas turbulence at said sensor to enhance sensor performance.

4. An environment sampling system according to claim 1 wherein said sensor further comprises a saddle support flange interface to support said sensor to mount therethrough said sidewall.

5. An environment sampling system according to claim 1 further comprising a means to regulate said gas movement via a gas pressure change and a gas flow rate change.

6. An environment sampling system according to claim 5 wherein said means to regulate said gas movement is a control valve having an adjustable range from a fully closed operational state to a fully open operational state.

7. An environment sampling system according to claim 6 wherein said control valve is disposed within said outlet portion being positioned downstream in relation to said gas movement from said means for initiating gas movement, this is to operationally regulate said gas movement via said gas pressure change and said gas flow rate change within said interior via moving along a performance curve of said means for initiating gas movement as required for said sensor to have enhanced gas contaminant detection performance by regulating said control valve between said fully closed state and said fully open state.

8. An environment sampling system according to claim 1 further comprising a controller that is in electrical communication with said sensor, wherein operationally said controller converts an analog signal input from said sensor into a digital output signal.

9. An environment sampling system according to claim 8 wherein said controller is positionally affixed to said sidewall in the atmospheric area to minimize distance of said sensor electrical communication, wherein a plurality of sensors would be facilitated to be disposed therethrough said sidewall.
